(19) United States
(12) Patent Application Publication
(10) Pub. No.: US 2019/0115110 A1
(43) Pub. Date: Apr. 18, 2019

(54) Systems and Methods for Medical Products and Services

(71) Applicant: American TelePhysicians, Jacksonville, FL (US)

(72) Inventors: Waqas Ahmed, Jacksonville, FL (US); Junaid Ahmad, Dubai (AE); Naseer Khan, Mason, OH (US)

(21) Appl. No.: 16/159,389

(22) Filed: Oct. 12, 2018

Related U.S. Application Data
(60) Provisional application No. 62/572,296, filed on Oct. 13, 2017.

(51) Int. Cl. G16H 70/40; G16H 20/10; G16H 40/60

(52) U.S. Cl. CPC G16H 70/40; G16H 50/20; G16H 40/60; G16H 20/10

(57) ABSTRACT

Provided herein is a system including, in some embodiments, one or more web servers, one or more application servers, and one or more database servers configured to provide one-stop healthcare to users through an online portal in at least cash-based health markets. The one or more web servers are configured to accept electronic requests from web clients for access to the online portal for different healthcare-related products and services. The one or more application servers are configured to provide the online portal to the web clients on demand. The one or more database servers include a database management system configured to store one or more vendors and the vendors' prices for the different healthcare-related products and services. The system is configured to provide the one or more vendors and the vendors' prices for the different healthcare-related products and services through the online portal to the web clients on demand.

### PRIORITY

This application claims the benefit of priority to U.S. Provisional Patent Application No. 62/572,296, filed Oct. 13, 2017, titled, “SYSTEMS AND METHODS FOR MEDICAL PRODUCTS AND SERVICES,” which is incorporated by reference in its entirety into this application.

### BACKGROUND

Cash-based health markets in countries such as India, Mexico, Pakistan, Nigeria, and the like currently suffer from a number of problems including poorly integrated healthcare systems, a lack of integrated primary care physician services, a lack of transparency and healthcare information, a lack of good home care services, and inaccessibility to quality healthcare. Regarding poorly integrated healthcare systems, the healthcare systems in these countries are poorly integrated, making healthcare service acquisition a hectic, time consuming, and frustrating affair. Regarding the lack of primary care physician services, unlike the first world, primary healthcare physician services in these parts of the world are not well established, which results in poor patient education and follow up. Regarding the lack of transparency and healthcare information, owing to various factors, the availability of information about healthcare is very scarce. Most consumers do not know where to look for such information since official information is often unavailable, outdated, or vague. Regarding the lack of home care services, the availability of homecare services like home physiotherapy and home nursing is poor in these countries. When in need, consumers often do not know where to find such homecare services—even if available. Regarding inaccessibility to quality healthcare, despite having a financial means, consumers often do not have access to quality healthcare services owing to logistical limitations, procedural complications, and visa hurdles. Moreover, healthcare costs are not transparent in these markets.

In addition to the foregoing number of problems in cash-based health markets, some existing health markets are not meaningfully accessible to certain segments of the population. For example, certain segments of the population in the United States have either high-deductible health insurance or no health insurance at all. Moreover, there are a number of predominantly cash-based healthcare services such as plastic surgery, cosmetic dental procedures, and the like, but there is no comprehensive platform to consolidate such healthcare services geographically and on an as-needed basis.

As such, provided herein are systems and methods for medical products and services that meet or exceed the foregoing needs.

### SUMMARY

Provided herein is a system including, in some embodiments, one or more web servers, one or more application servers, and one or more database servers configured to provide one-stop healthcare to a number of users through an online portal in at least cash-based health markets. The one or more web servers are configured to accept electronic requests from a number of web clients for access to the online portal for different healthcare-related products and services. The one or more application servers are configured to provide the online portal to the web clients on demand. The one or more database servers include a database management system configured to store one or more vendors and the vendors' prices for the different healthcare-related products and services. The system is configured to provide the one or more vendors and the vendors' prices for the different healthcare-related products and services through the online portal to the web clients on demand.

In some embodiments, the healthcare-related products and services include one or more products and services selected from lab tests, imaging, medical packages, automated diagnoses, appointment scheduling, tele-medicine, home care, online pharmacy services, and second opinions.

In some embodiments, the healthcare-related products and services include lab tests, the online portal providing price comparisons of different lab tests, home sampling options, home or online delivery of lab test results, or a combination thereof

In some embodiments, the healthcare-related products and services include imaging, the online portal providing price comparisons of different imaging procedures, online scheduling, home or online delivery of imaging results, or a combination thereof

In some embodiments, the healthcare-related products and services include medical packages, the online portal providing symptoms-based medical packages with lab tests for purchase as recommended by top-tier physicians.

In some embodiments, the healthcare-related products and services include automated diagnoses, the online portal providing diagnoses or recommendations with artificial intelligence-based technology and algorithms designed by top-tier physicians.

In some embodiments, the healthcare-related products and services include appointment scheduling, the online portal providing appointments with local or remote physicians or other healthcare professionals.

In some embodiments, the healthcare-related products and services include tele-medicine, the online portal providing live consultation with one or more top-tier physicians, dieticians, physiotherapists, speech therapists, or psychologists.

In some embodiments, the healthcare-related products and services include home-based care, the online portal providing information for home physiotherapy, home speech therapy, or home doctors, as well as scheduling such home-based care online.

In some embodiments, the healthcare-related products and services include online pharmacy services, the online portal providing online ordering and home delivery of medicaments.

In some embodiments, the healthcare-related products and services include second medical opinions, the online portal providing on-demand access to top-tier physicians for the second medical opinions.

Also provided herein is one or more non-transitory computer-readable media configured to store instructions that, when executed by one or more processors of a system, cause the system to perform operations including, in some embodiments, accepting electronic requests by one or more web servers of the system, the electronic requests from a number of web clients for access to an online portal for different healthcare-related products and services; providing the online portal to the web clients on demand by one or more application servers of the system; storing one or more vendors and vendors' prices for the different healthcare-related products and services, the one or more vendors and the vendors' prices stored by one or more database servers including a database management system; and providing the one or more vendors and the vendors' prices for the different healthcare-related products and services to the web clients on demand. As such, the one or more non-transitory computer-readable media configures the system to provide one-stop healthcare through the online portal, for example, in at least cash-based health markets.

In some embodiments, the healthcare-related products and services provided by the online portal include one or more products and services selected from lab tests, imaging, medical packages, automated diagnoses, appointment scheduling, tele-medicine, home care, online pharmacy services, and second opinions.

In some embodiments, the healthcare-related products and services provided by the online portal include lab tests, the online portal providing price comparisons of different lab tests, home sampling options, home or online delivery of lab test results, or a combination thereof

In some embodiments, the healthcare-related products and services provided by the online portal include imaging, the online portal providing price comparisons of different imaging procedures, online scheduling, home or online delivery of imaging results, or a combination thereof

In some embodiments, the healthcare-related products and services provided by the online portal include medical packages, the online portal providing symptoms-based medical packages with lab tests for purchase as recommended by top-tier physicians.

In some embodiments, the healthcare-related products and services provided by the online portal include automated diagnoses, the online portal providing diagnoses or recommendations with artificial intelligence-based technology and algorithms designed by top-tier physicians.

In some embodiments, the healthcare-related products and services provided by the online portal include appointment scheduling, the online portal providing appointments with local or remote physicians or other healthcare professionals.

In some embodiments, the healthcare-related products and services provided by the online portal include tele-medicine, the online portal providing live consultation with one or more top-tier physicians, dieticians, physiotherapists, speech therapists, or psychologists.

In some embodiments, the healthcare-related products and services provided by the online portal include home-based care, the online portal providing information for home physiotherapy, home speech therapy, or home doctors, as well as scheduling such home-based care online.

In some embodiments, the healthcare-related products and services provided by the online portal include online pharmacy services, the online portal providing online ordering and home delivery of medicaments.

In some embodiments, the healthcare-related products and services provided by the online portal include second medical opinions, the online portal providing on-demand access to top-tier physicians for the second medical opinions.

### DRAWINGS

FIG. 1 provides a schematic illustrating parties in collaboration to provide medical services and products to a number of users, or patients, in accordance with some embodiments.

FIG. 2A provides an image of a first view of a web-based portal configured to provide medical services and products to a number of users in accordance with some embodiments.

FIG. 2B provides an image of a second view of a web-based portal configured to provide medical services and products to a number of users in accordance with some embodiments.

FIG. 2C provides an image of a third view of a web-based portal configured to provide medical services and products to a number of users in accordance with some embodiments.

FIG. 2D provides an image of a fourth view of a web-based portal configured to provide medical services and products to a number of users in accordance with some embodiments.

FIG. 2E provides an image of a fifth view of a web-based portal configured to provide medical services and products to a number of users in accordance with some embodiments.

FIG. 2F provides an image of a sixth view of a web-based portal configured to provide medical services and products to a number of users in accordance with some embodiments.

FIG. 3A provides an image of a first view of a mobile-device portal of healthcare-related products and services in accordance with some embodiments.

FIG. 3B provides an image of a second view of a mobile-device portal of healthcare-related products and services in accordance with some embodiments.

FIG. 3C provides an image of a third view of a mobile-device portal of healthcare-related products and services in accordance with some embodiments.

FIG. 3D provides an image of a fourth view of a mobile-device portal of healthcare-related products and services in accordance with some embodiments.

FIG. 3E provides an image of a fifth view of a mobile-device portal of healthcare-related products and services in accordance with some embodiments.

FIG. 3F provides an image of a sixth view of a mobile-device portal of healthcare-related products and services in accordance with some embodiments.

FIG. 3G provides an image of a seventh view of a mobile-device portal of healthcare-related products and services in accordance with some embodiments.

FIG. 3H provides an image of an eighth view of a mobile-device portal of healthcare-related products and services in accordance with some embodiments.

FIG. 3I provides an image of a ninth view of a mobile-device portal of healthcare-related products and services in accordance with some embodiments.

FIG. 3J provides an image of a tenth view of a mobile-device portal of healthcare-related products and services in accordance with some embodiments.

FIG. 4A provides a schematic illustrating a system configured to provide a web-based portal to a desktop user in accordance with some embodiments.

FIG. 4B provides a schematic illustrating a system configured to provide a web-based portal to a mobile user in accordance with some embodiments.

FIG. 5 provides a schematic illustrating a computing system in accordance with some embodiments.

### DESCRIPTION

Before some particular embodiments are provided in greater detail, it should be understood that the particular embodiments provided herein do not limit the scope of the concepts provided herein. It should also be understood that a particular embodiment provided herein can have features that can be readily separated from the particular embodiment and optionally combined with or substituted for features of any of a number of other embodiments provided herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth herein above, cash-based health markets in countries such as India, Mexico, Pakistan, Nigeria, and the like currently suffer from a number of problems including poorly integrated healthcare systems; a lack of integrated primary care physician services; a lack of transparency and healthcare information; a lack of good home care services; and inaccessibility to quality healthcare. In addition to the foregoing number of problems in cash-based health markets, some existing health markets are not meaningfully accessible to certain segments of the population. For example, certain segments of the population in the United States have either high-deductible health insurance or no health insurance at all. Moreover, there are a number of predominantly cash-based healthcare services such as plastic surgery, cosmetic dental procedures, and the like, and there is no comprehensive platform to consolidate such healthcare services geographically and on an as-needed basis. As such, provided herein are systems and methods for medical products and services that meet or exceed the foregoing needs.

For example, a system is provided including, in some embodiments, one or more web servers, one or more application servers, and one or more database servers configured to provide one-stop healthcare to a number of users through an online portal in at least cash-based health markets. The one or more web servers are configured to accept electronic requests from a number of web clients for access to the online portal for different healthcare-related products and services. The one or more application servers are configured to provide the online portal to the web clients on demand. The one or more database servers include a database management system configured to store one or more vendors and the vendors' prices for the different healthcare-related products and services. The system is configured to provide the one or more vendors and the vendors' prices for the different healthcare-related products and services through the online portal to the web clients on demand.

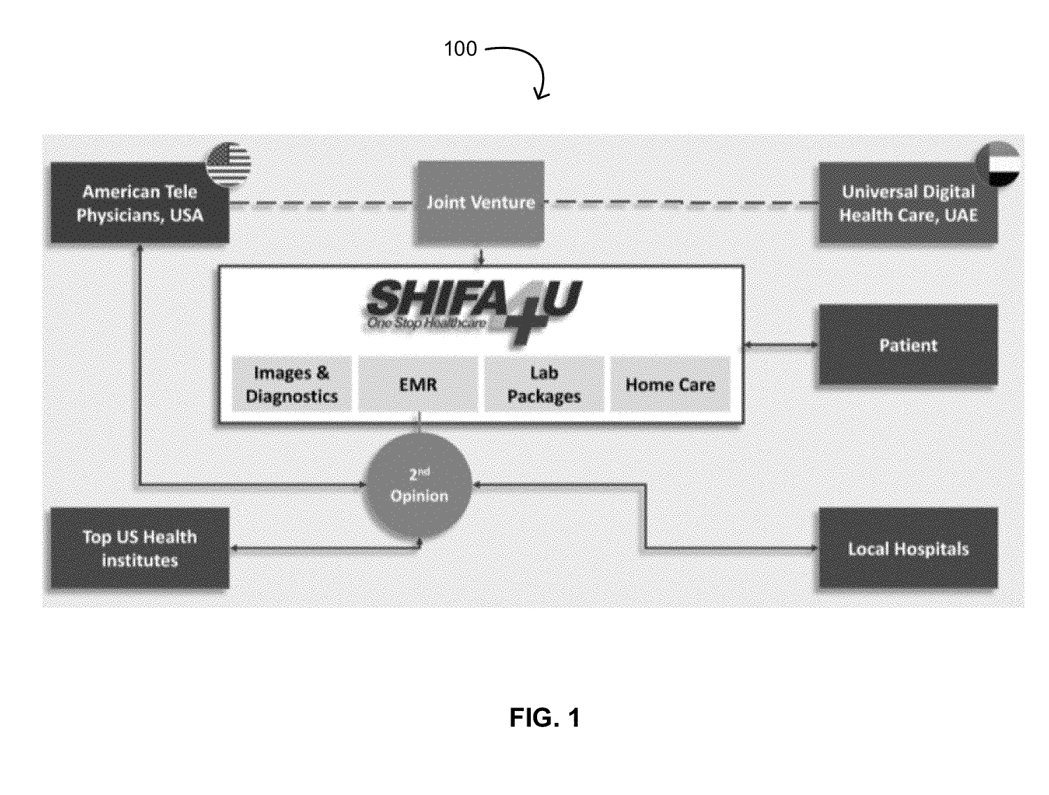

FIG. 1 provides a schematic 100 illustrating parties in a collaboration to provide medical services and products to a number of users, or patients, in accordance with some embodiments. As shown, the medical services and products can be provided through an online portal configured to provide one-stop healthcare to a number of users such as users in at least cash-based health markets. The collaboration can include one or more portal-collaborating parties, one or more medical service-collaborating parties, one or more medical product-collaborating parties, or a combination thereof. The one or more portal-collaborating parties can include, but is not limited to, a software developer. The one or more medical service-collaborating parties can include, but is not limited to, a selection of medical service-collaborating parties from hospitals, health institutes, and medical-service vendors. The one or more medical product-collaborating parties can include, but is not limited to, medical-product vendors. The online portal is configured to be compliant with privacy standards of the Health Insurance Portability and Accountability Act (“HIPAA”) of 1996 at least for users or patients in the United States, thereby keeping the users' or the patients' data private and secure.

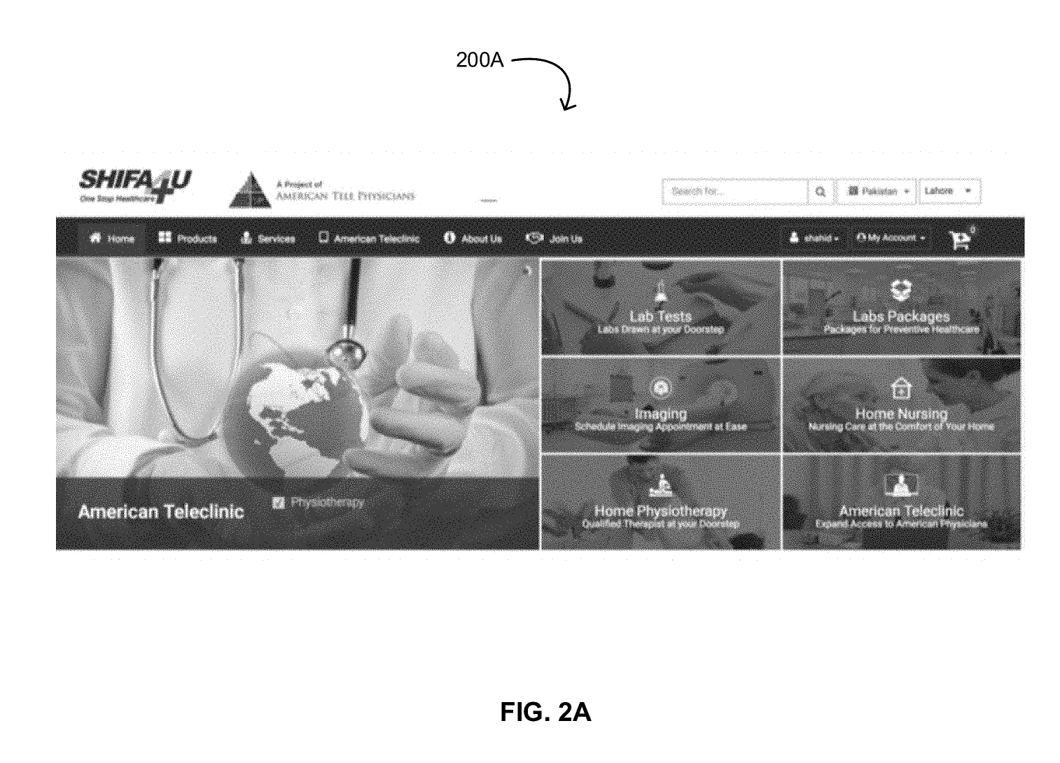

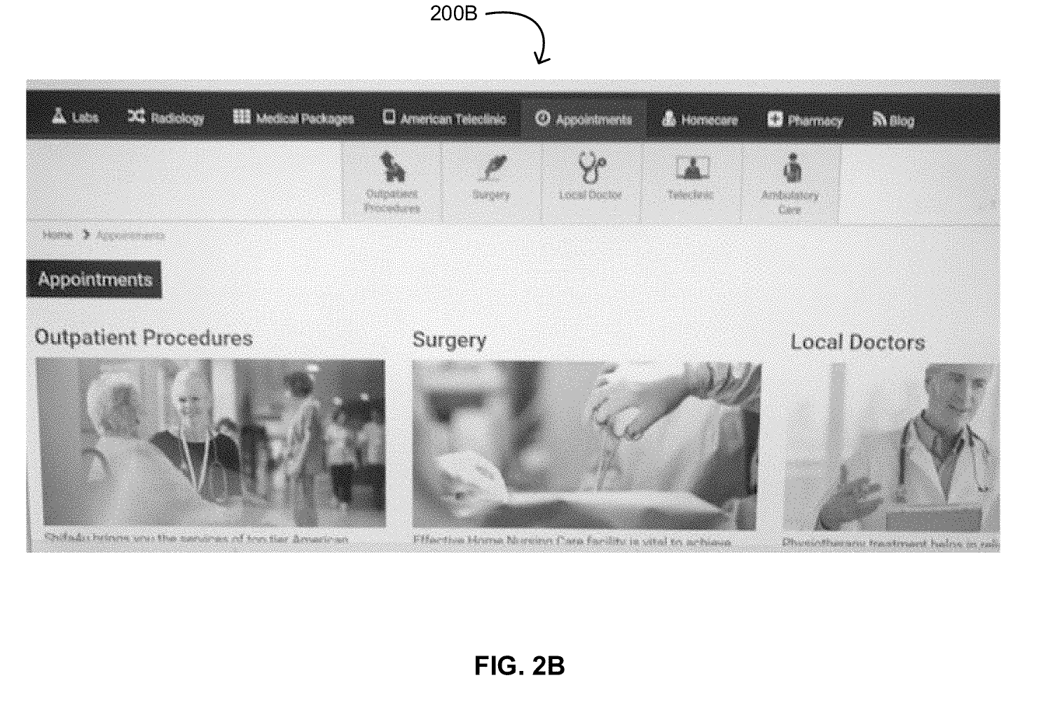

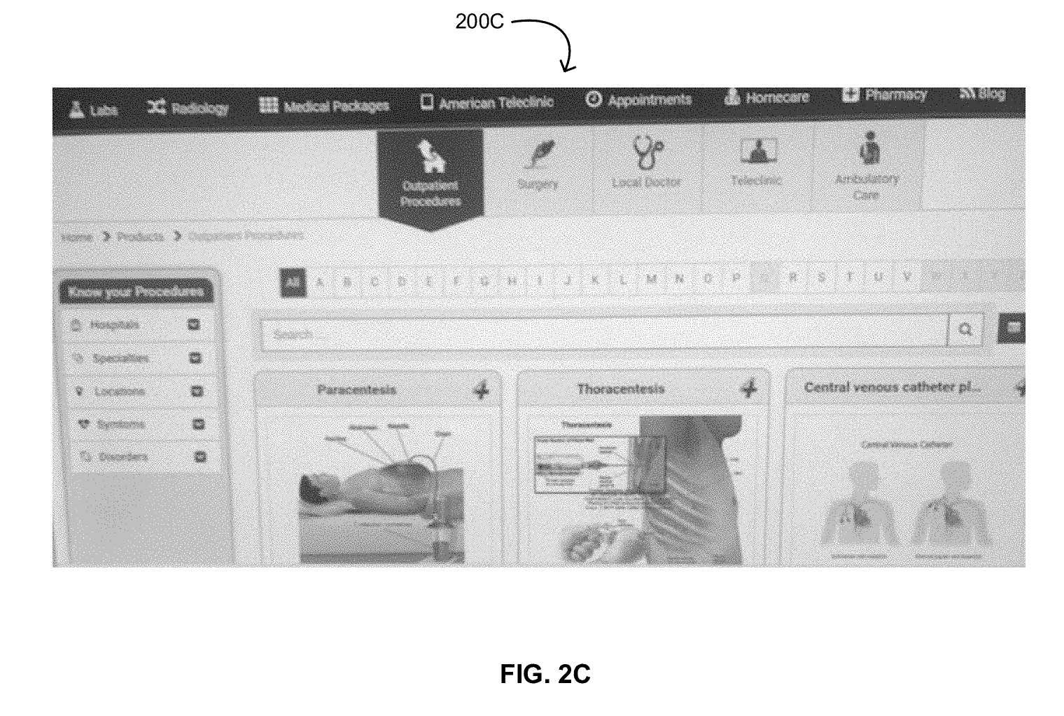

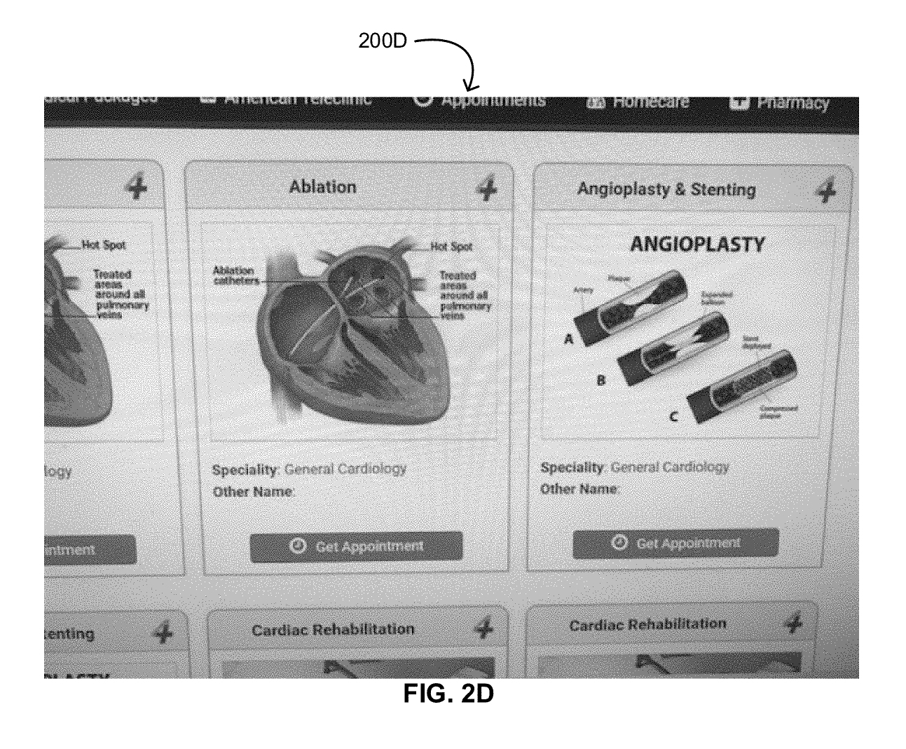

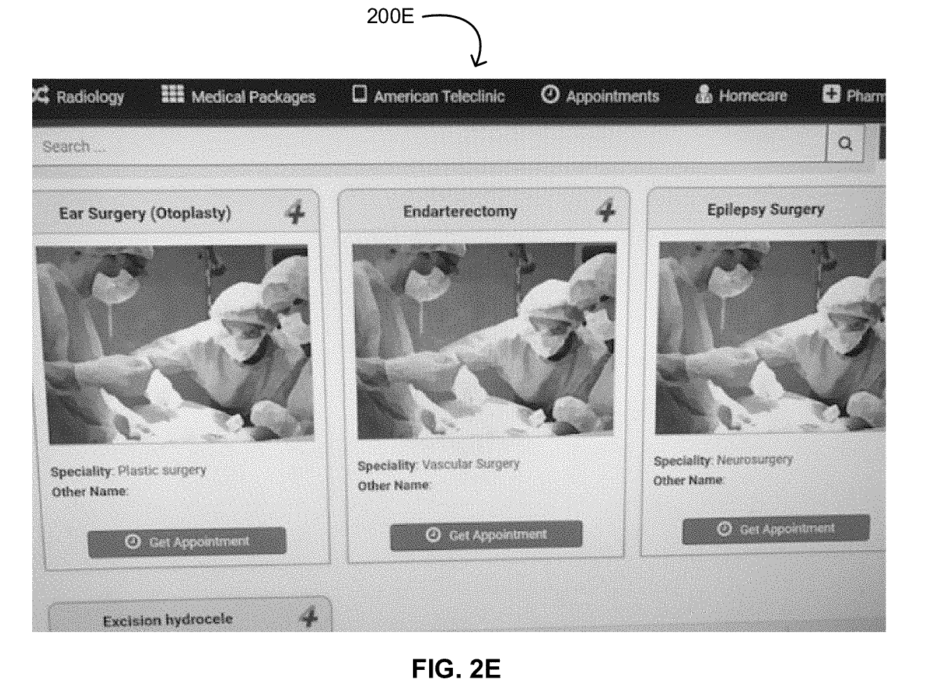

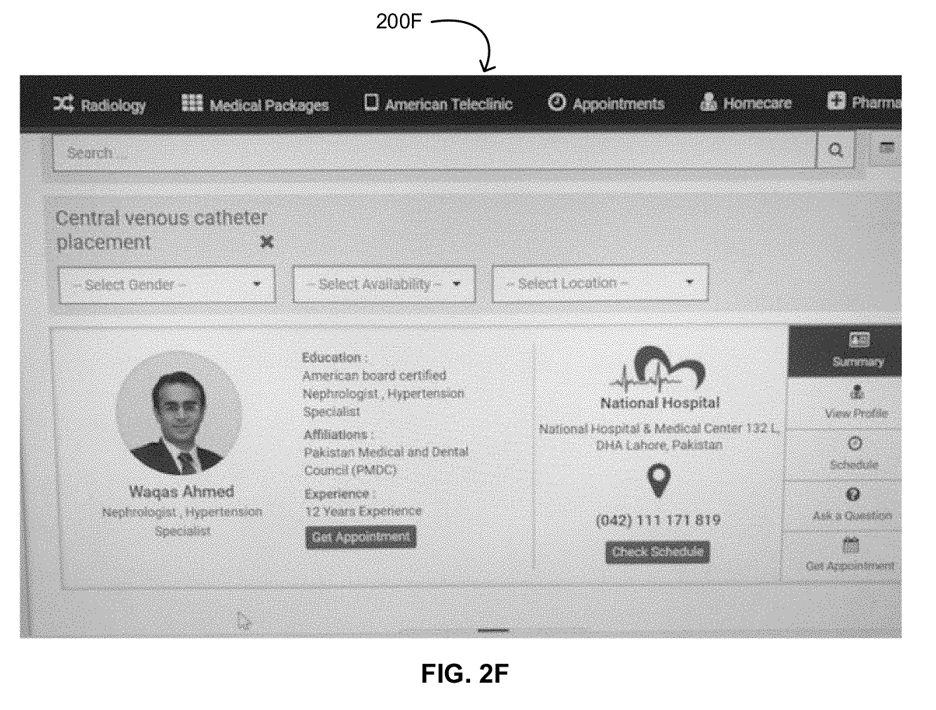

FIG. 2A provides an image 200A of a web-based portal configured to provide medical services and products to a number of users in accordance with some embodiments. As shown, the online portal for providing one-stop healthcare to a number of users in at least cash-based health markets can be a web-based portal. The web-based portal is configured to be accessible by way of a web browser on a computing device including, but not limited to, a computing device selected from desktop computing devices, mobile computing devices, and computing devices therebetween such as portable computing devices (e.g., laptop or notebook computers). The healthcare-related products and services of the web-based portal include one or more products and services selected from lab tests, imaging, medical packages, automated diagnoses, appointment scheduling, tele-medicine, home care, online pharmacy services, and second opinions. The web-based portal directly connects the number of users to such healthcare-related products and services in their geographic area or via tele-medicine in real time.

FIG. 2B provides an image 200B of a second view of the web-based portal configured to provide medical services and products to a number of users in accordance with some embodiments. As shown, the healthcare-related products and services include appointments for outpatient procedures, surgeries, local doctors, tele-medicine, and ambulatory care.

FIG. 2C provides an image 200C of a third view of the web-based portal configured to provide medical services and products to a number of users in accordance with some embodiments. As shown, the healthcare-related products and services include appointments for outpatient procedures including, but not limited to, paracentesis, thoracentesis, central venous catheter placement, and the like.

FIG. 2D provides an image 200D of a fourth view of the web-based portal configured to provide medical services and products to a number of users in accordance with some embodiments. As shown, the healthcare-related products and services include appointments for surgery including, but not limited to, ablation and angioplasty with stenting.

FIG. 2E provides an image 200E of a fifth view of the web-based portal configured to provide medical services and products to a number of users in accordance with some embodiments. As shown, the healthcare-related products and services include appointments for ear surgery or otoplasty, endarterectomy, and epilepsy surgery.

FIG. 2F provides an image 200F of a sixth view of the web-based portal configured to provide medical services and products to a number of users in accordance with some embodiments. As shown, the healthcare-related products and services include information for local doctors or other contracting health care professionals for making appointments with such doctors and health care professionals.

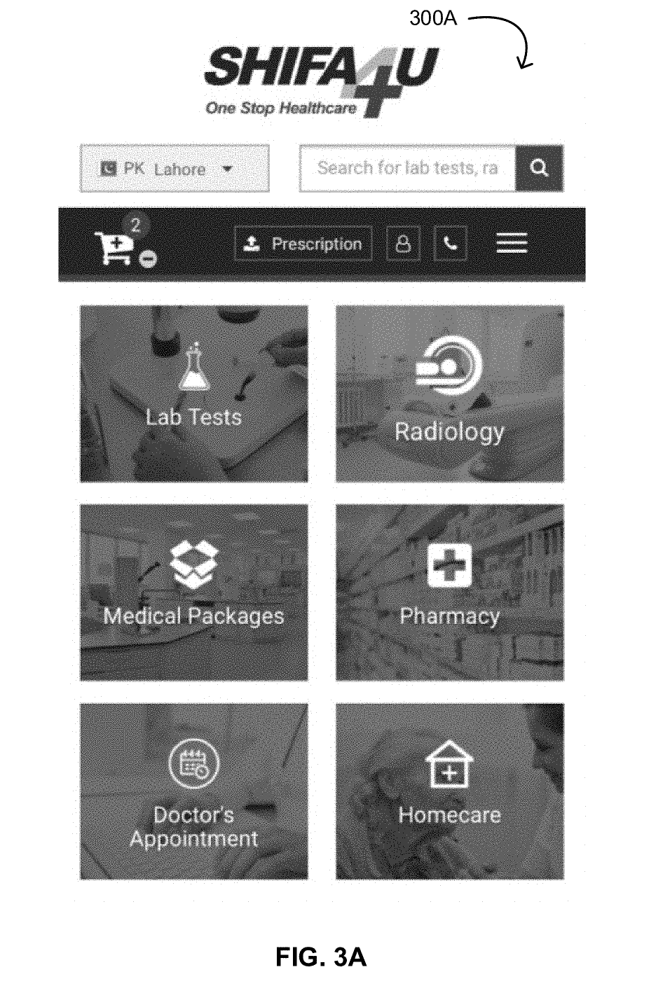

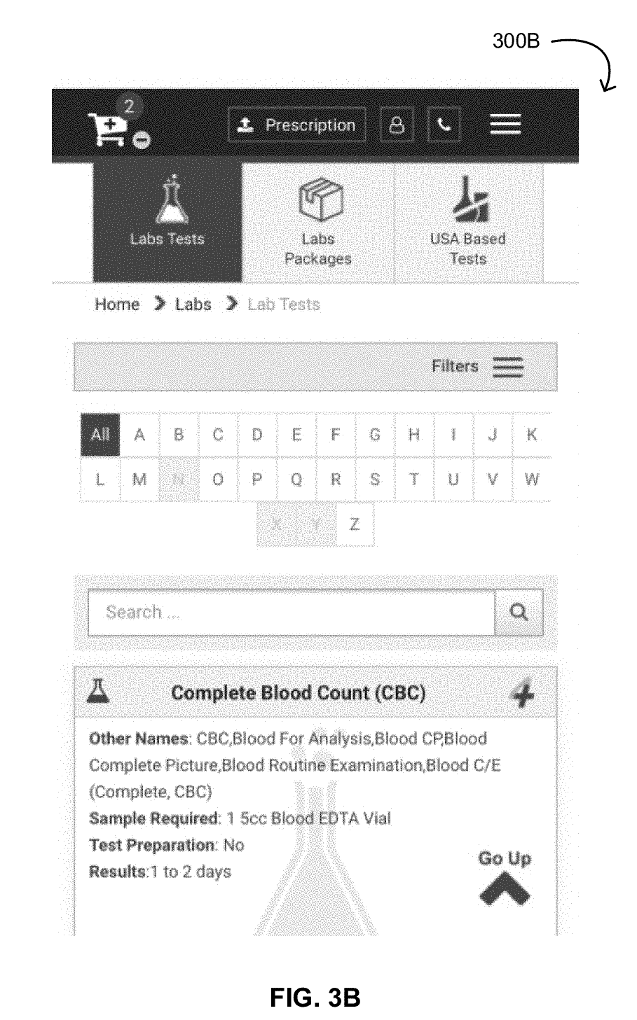

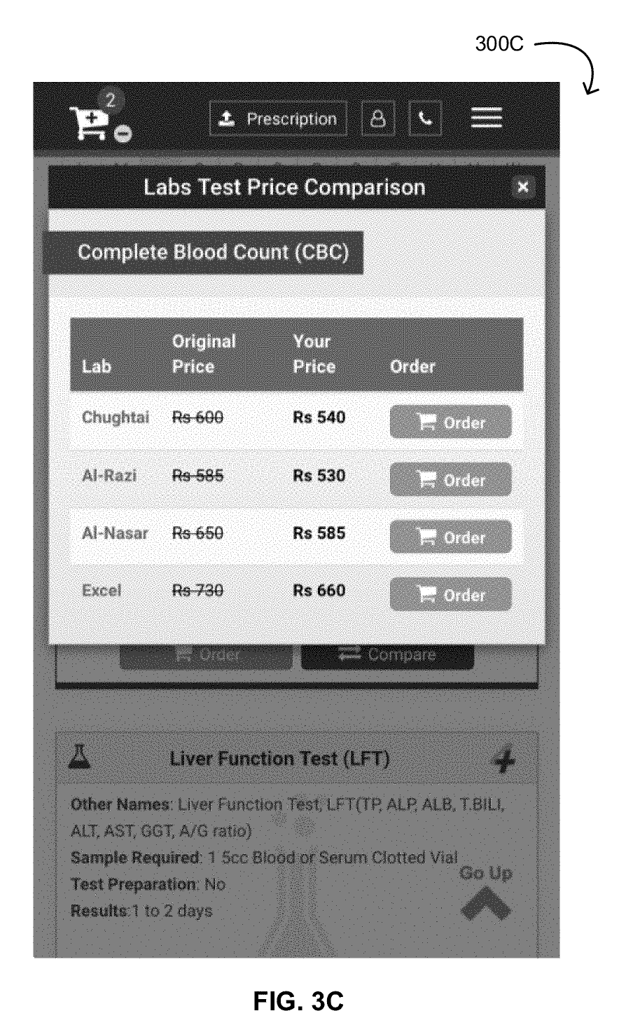

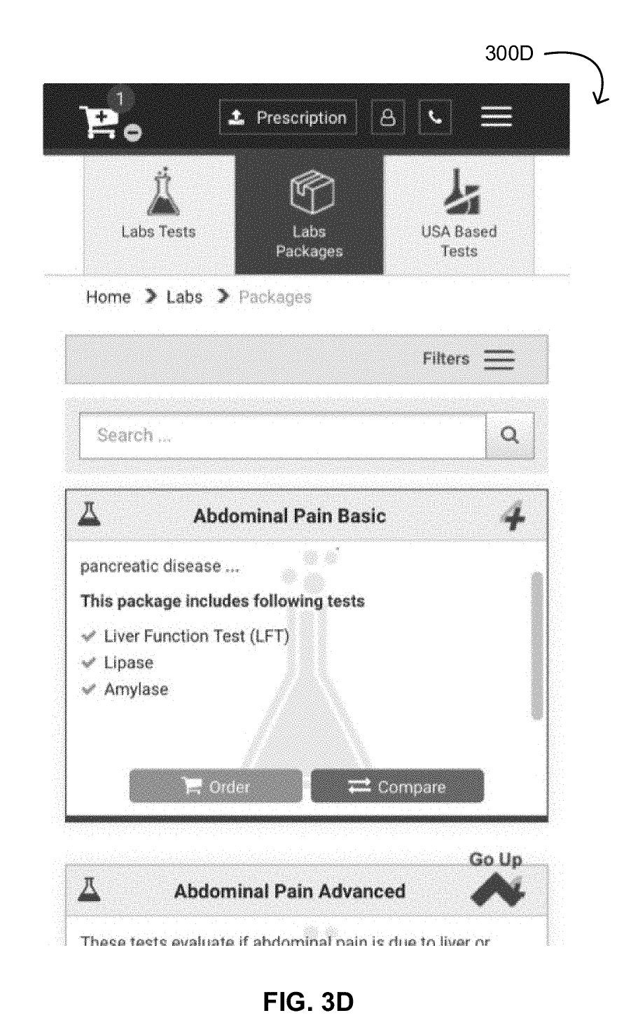

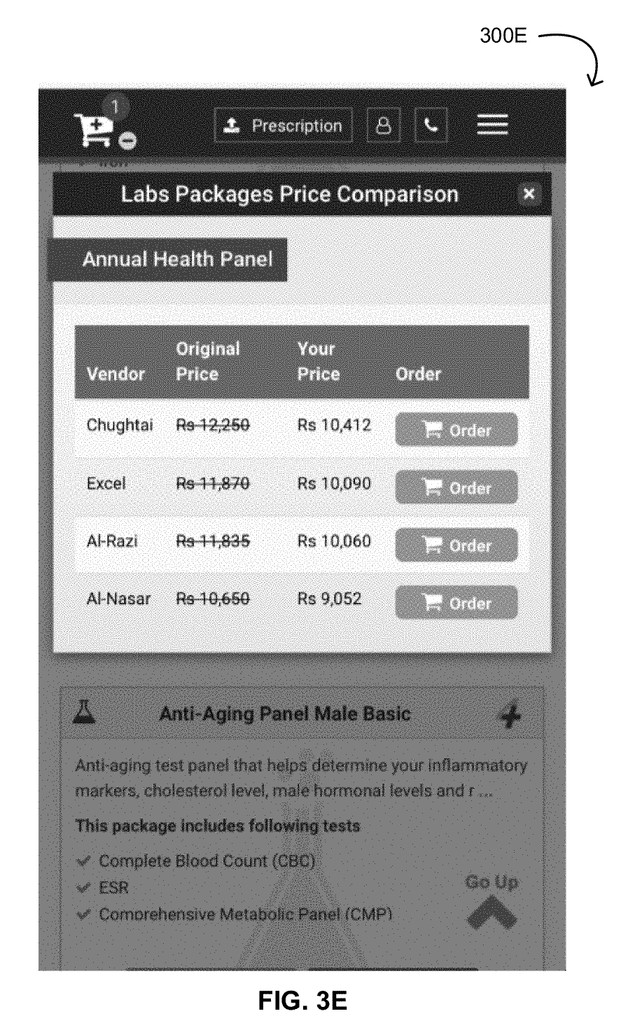

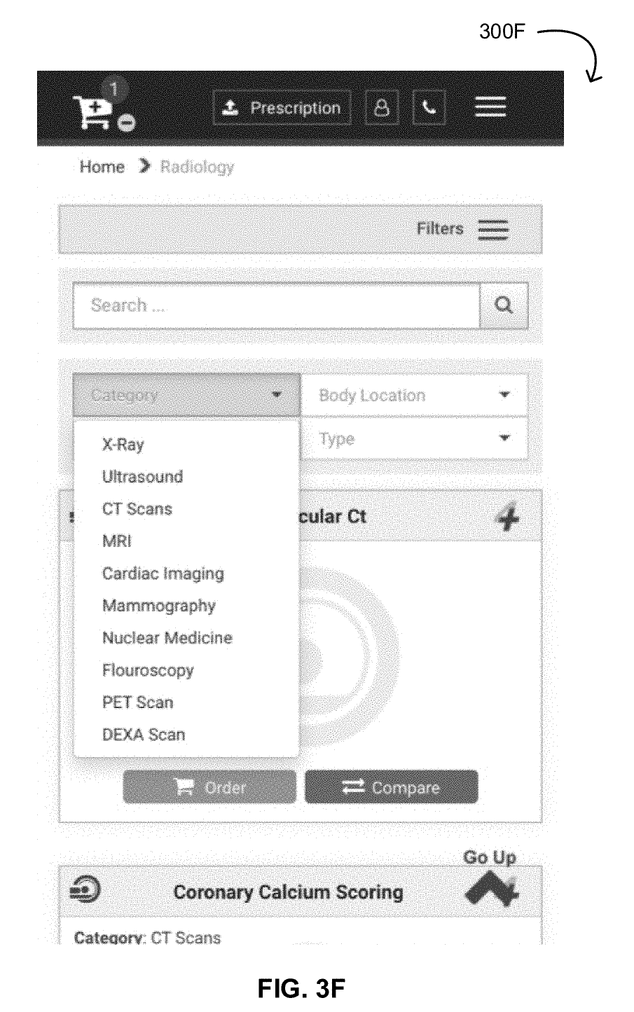

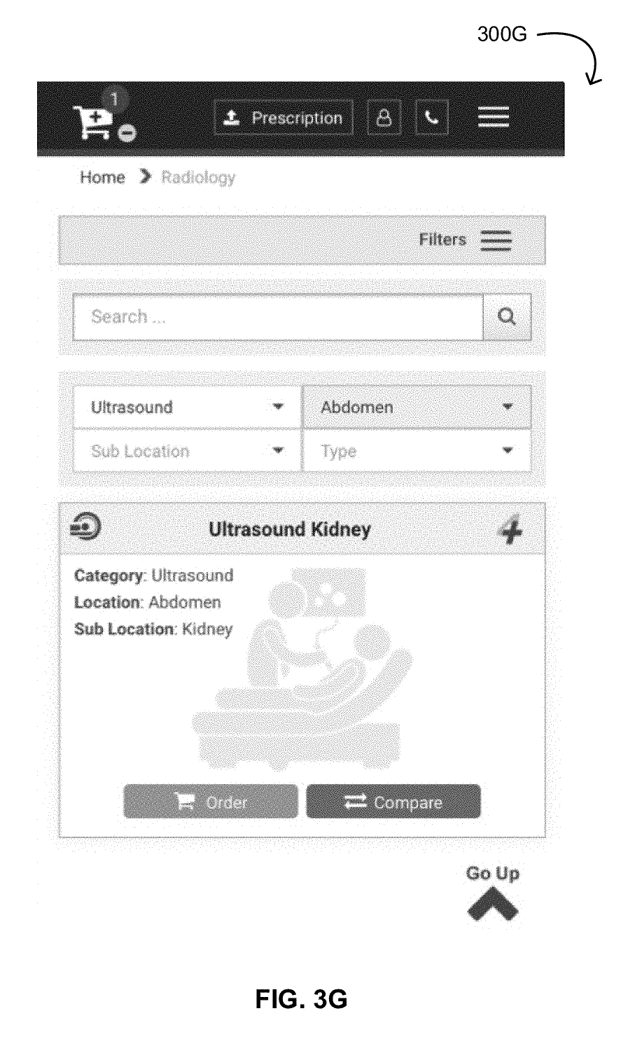

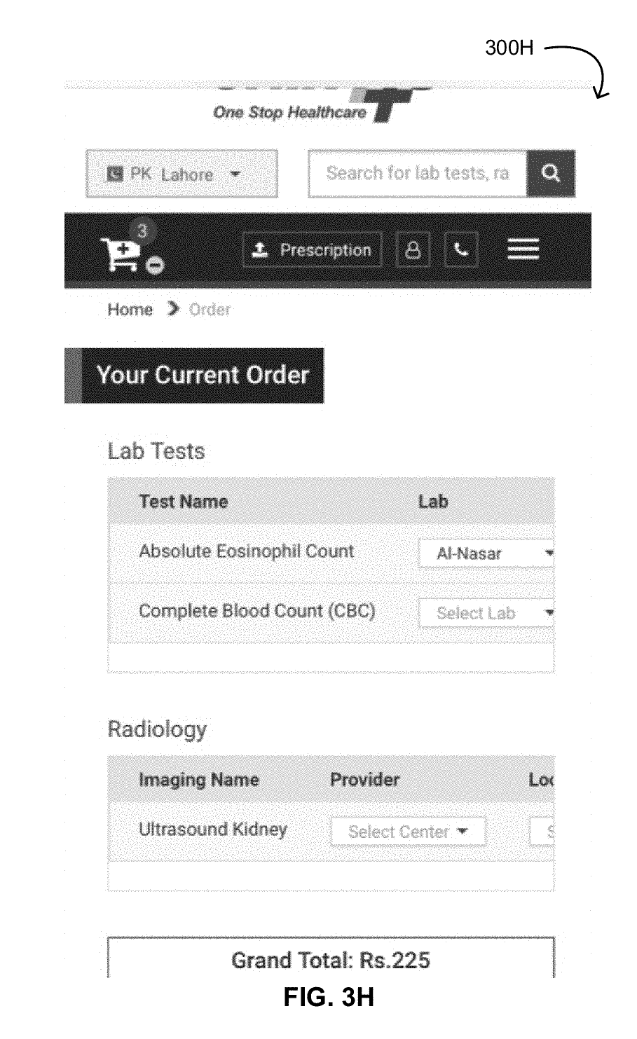

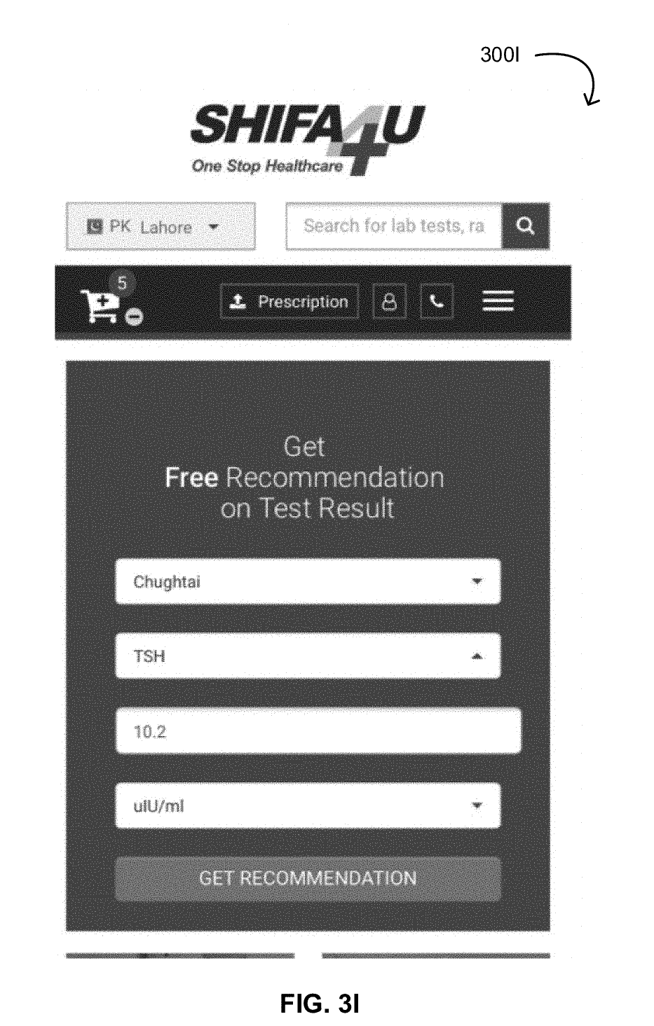

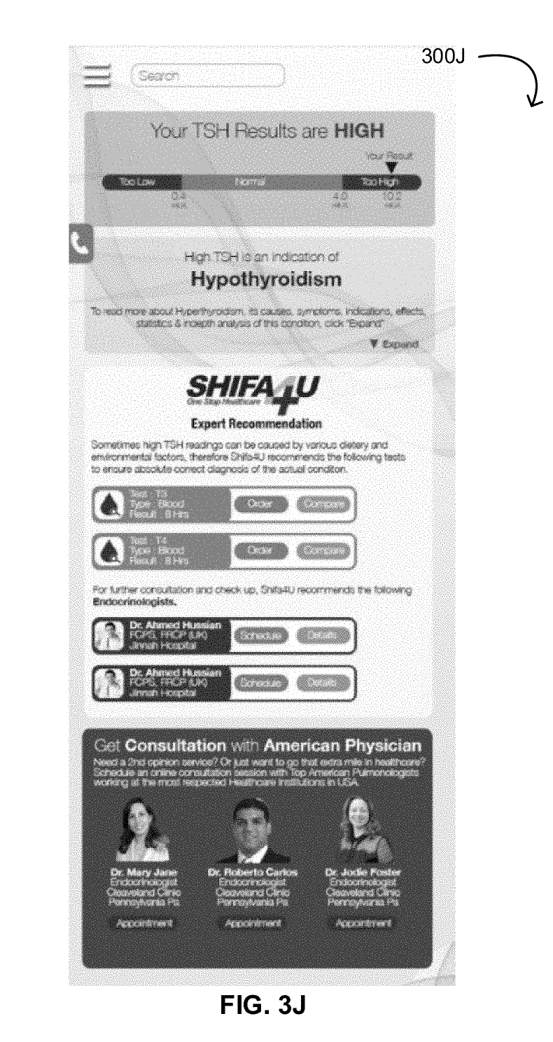

FIG. 3A provides an image 300A of a first view of the mobile-device portal of healthcare-related products and services in accordance with some embodiments. As shown, the mobile-device portal for providing one-stop healthcare to a number of users such in at least cash-based health markets can be a mobile-device portal. The mobile-device portal is configured to be accessible by way of an application native to a mobile computing device such as a mobile phone (e.g., smartphone) or a tablet computer. The healthcare-related products and services of the mobile-device portal include one or more products and services selected from lab tests, imaging, medical packages, automated diagnoses, appointment scheduling, tele-medicine, home care, online pharmacy services, and second opinions. The mobile-device portal directly connects the number of users to such healthcare-related products and services in their geographic area or via tele-medicine in real time.

Whether the online portal is a web-based portal or a mobile-device portal, the online portal is configured to provide similar, if not identical, healthcare-related products and services. In other words, similar, if not identical, healthcare-related products and services are provided through both the web-based portal and the mobile-device portal. As such, description of the healthcare-related products and services for the mobile-device portal also applies to the healthcare-related products and services for the web-based portal and vice versa.

FIG. 3B provides an image 300B of a second view of the mobile-device portal of healthcare-related products and services in accordance with some embodiments. FIG. 3C provides an image 300C of a third view of the mobile-device portal of healthcare-related products and services in accordance with some embodiments. As shown, the healthcare-related products and services include lab tests (e.g., Complete Blood Count [“CBC”]), the mobile-device portal providing price comparisons of different lab tests, home sampling options, home or online delivery of lab test results, or a combination thereof.

FIG. 3D provides an image 300D of a fourth view of the mobile-device portal of healthcare-related products and services in accordance with some embodiments. FIG. 3E provides an image 300E of a fifth view of the mobile-device portal of healthcare-related products and services in accordance with some embodiments. As shown, the healthcare-related products and services include medical packages, the mobile-device portal providing symptoms-based medical packages with lab tests for purchase. Such symptoms-based medical packages can include, for example, a basic abdominal pain package, which includes lab tests such as a Liver Function Test (“LFT”), as well as blood tests for key digestive enzymes such as lipase and amylase. The symptoms-based medical packages are those recommended by top-tier physicians including first-world physicians such as physicians from the United States or the United Kingdom. The mobile-device portal can be configured to provide price comparisons of the symptoms-based medical packages as shown in FIG. 3E.

FIG. 3F provides an image 300F of a sixth view of the mobile-device portal of healthcare-related products and services in accordance with some embodiments. FIG. 3G provides an image 300G of a seventh view of the mobile-device portal of healthcare-related products and services in accordance with some embodiments. As shown, the healthcare-related products and services include imaging (e.g., ultrasound imaging for a kidney), the mobile-device portal providing price comparisons of different imaging procedures, online scheduling, home or online delivery of imaging results, or a combination thereof

FIG. 3H provides an image 300H of an eighth view of a mobile-device portal of healthcare-related products and services in accordance with some embodiments. As shown, each of the healthcare-related products and services a user chooses (e.g., Absolute Eosinophil Count; Complete Blood Count; and the ultrasound imaging for the kidney) in the mobile-device portal can be added to a cart for checkout.

FIG. 3I provides an image 3001 of a ninth view of a mobile-device portal of healthcare-related products and services in accordance with some embodiments. As shown, the healthcare-related products and services include free recommendations on test results, optionally at a time of checkout for other healthcare-related products and services in a user's cart.

FIG. 3J provides an image 300J of a tenth view of a mobile-device portal of healthcare-related products and services in accordance with some embodiments. As shown, As shown, the healthcare-related products and services include appointments for recommendations, consultations, or both for test results. In this case, the test results indicate a high thyroid-stimulating hormone (“TSH”) value, and a number of doctors are presented in the mobile-device portal for the recommendations or consultations for the high TSH value.

The online portal can be further configured to provide any one or more of automated diagnoses, appointment scheduling, tele-medicine, home-based care, online pharmacy services, or second medical opinions. Automated diagnoses through the online portal includes providing diagnoses and recommendations with artificial intelligence-based technology and algorithms designed by top-tier physicians. The artificial intelligence-based technology and algorithms can be further configured to provide referral recommendations based on test results. (See FIG. 3J.) Appointment scheduling through the online portal includes providing appointments with local and remote physicians, surgeons, and other healthcare professionals. (See FIGS. 2B-2F.) With respect to surgeons, the online portal is configured to provide surgical procedures performed by the surgeons as well as providing appointments with the surgeons—even in remote locations. (See FIGS. 2C-2E.) Tele-medicine through the online portal includes providing live consultation with one or more top-tier physicians, dieticians, physiotherapists, speech therapists, or psychologists. In addition, the online portal is configured to allow patients with existing medical records, medical reports, lab results, and the like to upload such records, reports, and results for recommendations by way of the tele-medicine such as recommendations for further diagnostics or treatment by local physicians. Home-based care through the online portal includes providing information for home physiotherapy, home speech therapy, or home doctors, as well as scheduling such home-based care online. The online pharmacy services through the online portal include online ordering and home delivery of medicaments. The second medical opinions through the online portal include providing on-demand access to top-tier physicians for the second medical opinions.

In addition, the online portal can be further configured as a repository for electronic medical records. (See FIG. 1.) Such electronic medical records are configured to be accessible to users or patients and treatment-providing local and remote physicians, surgeons, and other healthcare professionals. The electronic medical records are configured to be accessible online, and orders for any tests, referrals, etc. can be placed in the electronic medical records in real time.

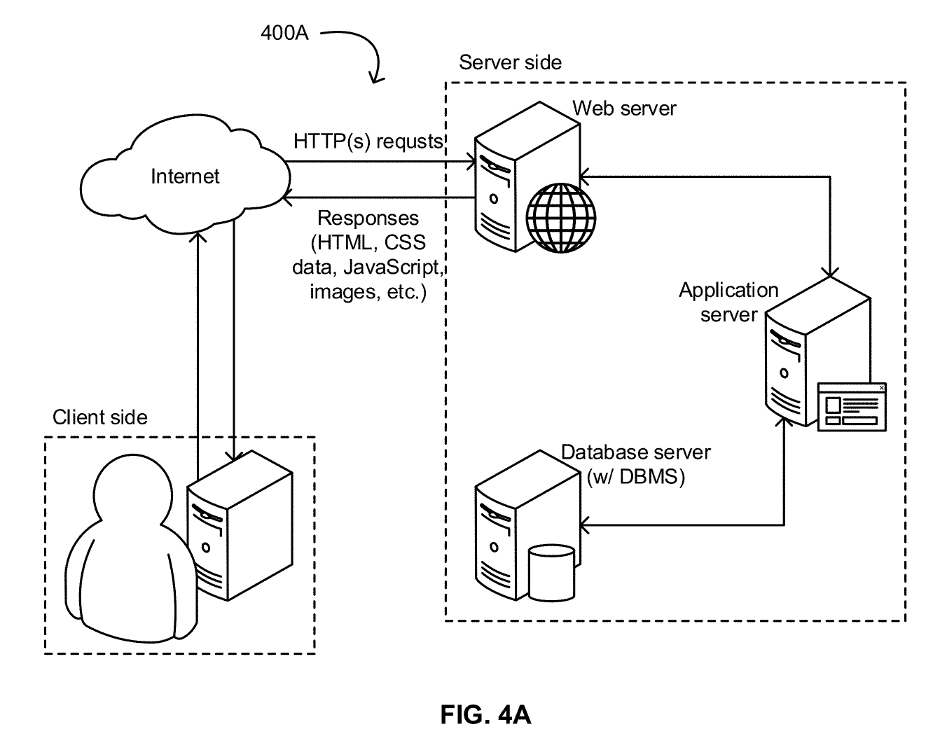

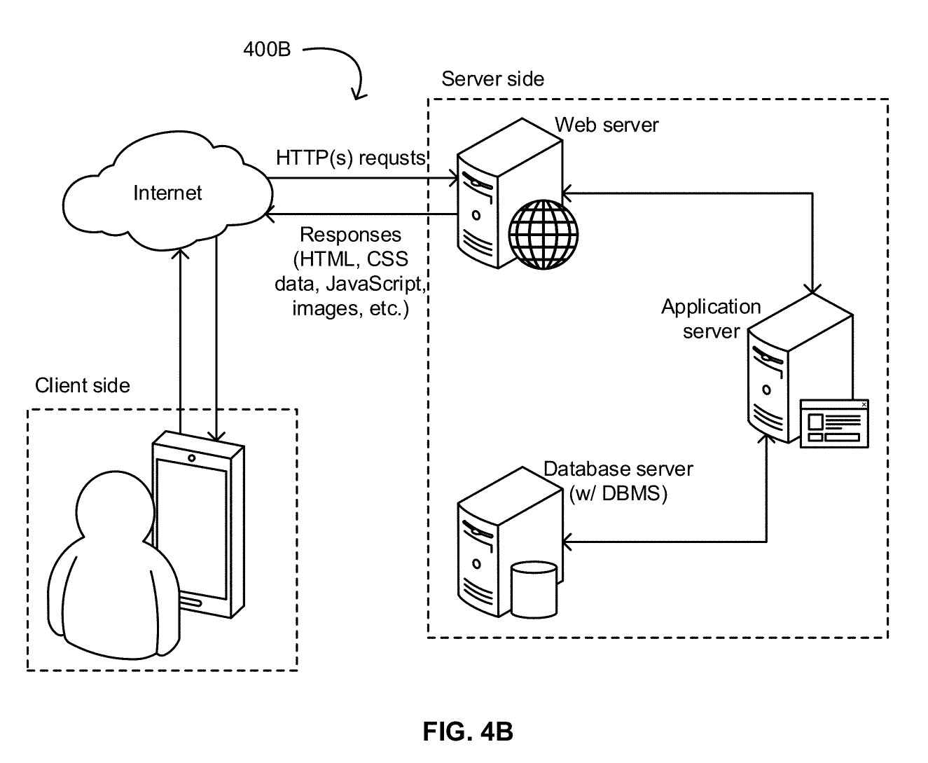

FIG. 4A provides a schematic illustrating a system 400A configured to provide a web-based portal to a desktop user in accordance with some embodiments. FIG. 4B provides a schematic illustrating a system 400B configured to provide a web-based portal to a mobile user in accordance with some embodiments. The system 400A and the system 400B can be the same system or different systems.

As shown, the system includes one or more web servers, one or more application servers, and one or more database servers on a server side of the system configured to provide one-stop healthcare to a number of users through an online portal in at least cash-based health markets. The one or more web servers are configured to accept electronic requests from a number of web clients for access to the online portal for different healthcare-related products and services. The one or more application servers are configured to provide the online portal to the web clients on demand. The one or more database servers include a database management system configured to store one or more vendors and the vendors' prices for the different healthcare-related products and services. The system is configured to provide the one or more vendors and the vendors' prices for the different healthcare-related products and services through the online portal to the web clients on demand.

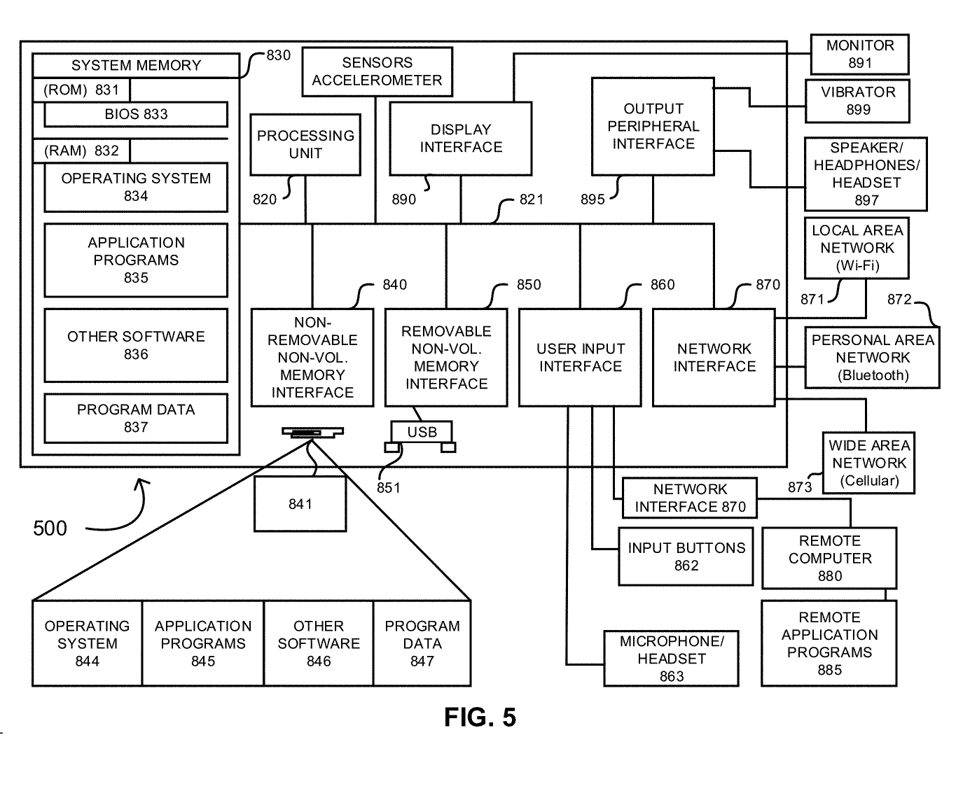

FIG. 5 provides a schematic illustrating a computing system 500 in accordance with some embodiments. The computing system, or any portion thereof, can be part of any one or more of the client or server computing devices provided herein. With reference to FIG. 5, components of the computing system 500 may include, but are not limited to, a processing unit 820 having one or more processing cores, a system memory 830, and a system bus 821 that couples various system components including the system memory 830 to the processing unit 820. The system bus 821 may be any of several types of bus structures selected from a memory bus or memory controller, a peripheral bus, and a local bus using any of a variety of bus architectures.

Computing system 500 typically includes a variety of computing machine-readable media. Computing machine-readable media can be any available media that can be accessed by computing system 500 and includes both volatile and nonvolatile media, and removable and non-removable media. By way of example, and not limitation, computing machine-readable media use includes storage of information, such as computer-readable instructions, data structures, other executable software or other data. Computer-storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium which can be used to store the desired information, and which can be accessed by the computing device 500. Transitory media such as wireless channels are not included in the machine-readable media. Communication media typically embody computer readable instructions, data structures, other executable software, or other transport mechanism and includes any information delivery media. As an example, some client computing systems on a network might not have optical or magnetic storage.

The system memory 830 includes computer storage media in the form of volatile and/or nonvolatile memory such as read only memory (ROM) 831 and random-access memory (RAM) 832. A basic input/output system 833 (BIOS) containing the basic routines that help to transfer information between elements within the computing system 500, such as during start-up, is typically stored in ROM 831. RAM 832 typically contains data and/or software that are immediately accessible to and/or presently being operated on by the processing unit 820. By way of example, and not limitation, FIG. 5 illustrates that RAM 832 can include a portion of the operating system 834, application programs 835, other executable software 836, and program data 837.

The computing system 500 can also include other removable/non-removable volatile/nonvolatile computer storage media. By way of example only, FIG. 5 illustrates a solid-state memory 841. Other removable/non-removable, volatile/nonvolatile computer storage media that can be used in the example operating environment include, but are not limited to, USB drives and devices, flash memory cards, solid state RAM, solid state ROM, and the like. The solid-state memory 841 is typically connected to the system bus 821 through a non-removable memory interface such as interface 840, and USB drive 851 is typically connected to the system bus 821 by a removable memory interface, such as interface 850.

The drives and their associated computer storage media discussed above and illustrated in FIG. 5, provide storage of computer readable instructions, data structures, other executable software and other data for the computing system 500. In FIG. 5, for example, the solid-state memory 841 is illustrated for storing operating system 844, application programs 845, other executable software 846, and program data 847. Note that these components can either be the same as or different from operating system 834, application programs 835, other executable software 836, and program data 837. Operating system 844, application programs 845, other executable software 846, and program data 847 are given different numbers here to illustrate that, at a minimum, they are different copies.

A user may enter commands and information into the computing system 500 through input devices such as a keyboard, touchscreen, or software or hardware input buttons 862, a microphone 863, a pointing device and/or scrolling input component, such as a mouse, trackball or touch pad. The microphone 863 can cooperate with speech recognition software. These and other input devices are often connected to the processing unit 820 through a user input interface 860 that is coupled to the system bus 821, but can be connected by other interface and bus structures, such as a parallel port, game port, or a universal serial bus (USB). A display monitor 891 or other type of display screen device is also connected to the system bus 821 via an interface, such as a display interface 890. In addition to the monitor 891, computing devices may also include other peripheral output devices such as speakers 897, a vibrator 899, and other output devices, which may be connected through an output peripheral interface 895.

The computing system 500 can operate in a networked environment using logical connections to one or more remote computers/client devices, such as a remote computing system 880. The remote computing system 880 can a personal computer, a hand-held device, a server, a router, a network PC, a peer device or other common network node, and typically includes many or all of the elements described above relative to the computing system 500. The logical connections depicted in FIG. 5 can include a personal area network (PAN) 872 (e.g., Bluetooth®), a local area network (LAN) 871 (e.g., Wi-Fi), and a wide area network (WAN) 873 (e.g., cellular network), but may also include other networks such as a personal area network (e.g., Bluetooth®). Such networking environments are commonplace in offices, enterprise-wide computer networks, intranets and the Internet. A browser application may be resident on the computing device and stored in the memory.

When used in a LAN networking environment, the computing system 500 is connected to the LAN 871 through a network interface or adapter 870, which can be, for example, a Bluetooth® or Wi-Fi adapter. When used in a WAN networking environment (e.g., Internet), the computing system 500 typically includes some means for establishing communications over the WAN 873. With respect to mobile telecommunication technologies, for example, a radio interface, which can be internal or external, can be connected to the system bus 821 via the network interface 870, or other appropriate mechanism. In a networked environment, other software depicted relative to the computing system 500, or portions thereof, may be stored in the remote memory storage device. By way of example, and not limitation, FIG. 5 illustrates remote application programs 885 as residing on remote computing device 880. It will be appreciated that the network connections shown are examples and other means of establishing a communications link between the computing devices may be used.

As discussed, the computing system 500 can include a processor 820, a memory (e.g., ROM 831, RAM 832, etc.), a built-in battery to power the computing device, an AC power input to charge the battery, a display screen, a built-in Wi-Fi circuitry to wirelessly communicate with a remote computing device connected to network.

It should be noted that the present design can be carried out on a computing system such as that described with respect to FIG. 5. However, the present design can be carried out on a server, a computing device devoted to message handling, or on a distributed system in which different portions of the present design are carried out on different parts of the distributed computing system.

Another device that may be coupled to bus 821 is a power supply such as a DC power supply (e.g., battery) or an AC adapter circuit. As discussed above, the DC power supply may be a battery, a fuel cell, or similar DC power source that needs to be recharged on a periodic basis. A wireless communication module can employ a Wireless Application Protocol to establish a wireless communication channel. The wireless communication module can implement a wireless networking standard.

In some embodiments, software used to facilitate algorithms discussed herein can be embodied onto a non-transitory machine-readable medium. A machine-readable medium includes any mechanism that stores information in a form readable by a machine (e.g., a computer). For example, a non-transitory machine-readable medium can include read only memory (ROM); random access memory (RAM); magnetic disk storage media; optical storage media; flash memory devices; Digital Versatile Disc (DVD's), EPROMs, EEPROMs, FLASH memory, magnetic or optical cards, or any type of media suitable for storing electronic instructions.

Note, an application described herein includes but is not limited to software applications, mobile applications, and programs that are part of an operating system application. Some portions of this description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. These algorithms can be written in a number of different software programming languages such as C, C+, or other similar languages. Also, an algorithm can be implemented with lines of code in software, configured logic gates in software, or a combination of both. In an embodiment, the logic consists of electronic circuits that follow the rules of Boolean Logic, software that contain patterns of instructions, or any combination of both.

It should be borne in mind, however, that all of these and similar terms are to be associated with the appropriate physical quantities and are merely convenient labels applied to these quantities. Unless specifically stated otherwise as apparent from the above discussions, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers, or other such information storage, transmission or display devices.

Many functions performed by electronic hardware components can be duplicated by software emulation. Thus, a software program written to accomplish those same functions can emulate the functionality of the hardware components in input-output circuitry.

While some particular embodiments have been provided herein, and while the particular embodiments have been provided in some detail, it is not the intention for the particular embodiments to limit the scope of the concepts presented herein. Additional adaptations and/or modifications can appear to those of ordinary skill in the art, and, in broader aspects, these adaptations and/or modifications are encompassed as well. Accordingly, departures may be made from the particular embodiments provided herein without departing from the scope of the concepts provided herein.

What is claimed is:

1. A system, comprising:
one or more web servers configured to accept electronic requests from a plurality of web clients for access to an online portal for different healthcare-related products and services;
one or more application servers configured to provide the online portal to the web clients on demand; and
one or more database servers including a database management system configured to store one or more vendors and the vendors' prices for the different healthcare-related products and services and provide the one or more vendors and the vendors' prices for the different healthcare-related products and services to the web clients on demand,
wherein the system is configured to provide one-stop healthcare through the online portal in at least cash-based health markets.

2. The system of claim 1, wherein the healthcare-related products and services include lab tests, the online portal providing price comparisons of different lab tests, home sampling options, home or online delivery of lab test results, or a combination thereof.

3. The system of claim 1, wherein the healthcare-related products and services include imaging, the online portal providing price comparisons of different imaging procedures, online scheduling, home or online delivery of imaging results, or a combination thereof

4. The system of claim 1, wherein the healthcare-related products and services include medical packages, the online portal providing symptoms-based medical packages with lab tests for purchase as recommended by top-tier physicians.

5. The system of claim 1, wherein the healthcare-related products and services include automated diagnoses, the online portal providing diagnoses or recommendations with artificial intelligence-based technology and algorithms designed by top-tier physicians.

6. The system of claim 1, wherein the healthcare-related products and services include appointment scheduling, the online portal providing appointments with local or remote physicians or other healthcare professionals.

7. The system of claim 1, wherein the healthcare-related products and services include tele-medicine, the online portal providing live consultation with one or more top-tier physicians, dieticians, physiotherapists, speech therapists, or psychologists.

8. The system of claim 1, wherein the healthcare-related products and services include home-based care, the online portal providing information for home physiotherapy, home speech therapy, or home doctors, as well as scheduling such home-based care online.

9. The system of claim 1, wherein the healthcare-related products and services include online pharmacy services, the online portal providing online ordering and home delivery of medicaments.

10. The system of claim 1, wherein the healthcare-related products and services include second medical opinions, the online portal providing on-demand access to top-tier physicians for the second medical opinions.

11. One or more non-transitory computer-readable media configured to store instructions that, when executed by one or more processors of a system, cause the system to perform operations, comprising:
accepting electronic requests by one or more web servers of the system, the electronic requests from a plurality of web clients for access to an online portal for different healthcare-related products and services;
providing the online portal to the web clients on demand by one or more application servers of the system;
storing one or more vendors and vendors' prices for the different healthcare-related products and services, the one or more vendors and the vendors' prices stored by one or more database servers including a database management system; and
providing the one or more vendors and the vendors' prices for the different healthcare-related products and services to the web clients on demand,
wherein the system is configured to provide one-stop healthcare through the online portal in at least cash-based health markets.

12. The one or more computer-readable media of claim 11, wherein the healthcare-related products and services provided by the online portal include lab tests, the online portal providing price comparisons of different lab tests, home sampling options, home or online delivery of lab test results, or a combination thereof.

13. The one or more computer-readable media of claim 11, wherein the healthcare-related products and services provided by the online portal include imaging, the online portal providing price comparisons of different imaging procedures, online scheduling, home or online delivery of imaging results, or a combination thereof

14. The one or more computer-readable media of claim 11, wherein the healthcare-related products and services provided by the online portal include medical packages, the online portal providing symptoms-based medical packages with lab tests for purchase as recommended by top-tier physicians.

15. The one or more computer-readable media of claim 11, wherein the healthcare-related products and services provided by the online portal include automated diagnoses, the online portal providing diagnoses or recommendations with artificial intelligence-based technology and algorithms designed by top-tier physicians.

16. The one or more computer-readable media of claim 11, wherein the healthcare-related products and services provided by the online portal include appointment scheduling, the online portal providing appointments with local or remote physicians or other healthcare professionals.

17. The one or more computer-readable media of claim 11, wherein the healthcare-related products and services provided by the online portal include tele-medicine, the online portal providing live consultation with one or more top-tier physicians, dieticians, physiotherapists, speech therapists, or psychologists.

18. The one or more computer-readable media of claim 11, wherein the healthcare-related products and services provided by the online portal include home-based care, the online portal providing information for home physiotherapy, home speech therapy, or home doctors, as well as scheduling such home-based care online.

19. The one or more computer-readable media of claim 11, wherein the healthcare-related products and services provided by the online portal include online pharmacy services, the online portal providing online ordering and home delivery of medicaments.

20. The one or more computer-readable media of claim 11, wherein the healthcare-related products and services provided by the online portal include second medical opinions, the online portal providing on-demand access to top-tier physicians for the second medical opinions.
